(19) United States
(12) Patent Application Publication
(10) Pub. No.: US 2026/0076721 A1
(43) Pub. Date: Mar. 19, 2026

(54) Modular Docking System for Arthroplasty and Fracture Implants

(71) Applicant: Matthew Klima, Tampa, FL (US)

(72) Inventor: Matthew Klima, Tampa, FL (US)

(21) Appl. No.: 19/281,603

(22) Filed: Jul. 26, 2025

(51) Int. Cl. A61B 17/72; A61B 17/00; A61B 17/56

(52) U.S. Cl. CPC A61B 17/72; A61B 2017/00486; A61B 2017/564

(57) ABSTRACT

A modular orthopedic implant system configured to interconnect fracture fixation implants with arthroplasty components across multiple anatomic sites. The system incorporates a clothespin-style tapered male stem and a fluted female receiver, forming a secure, fatigue-resistant mechanical interface. This docking feature enables modular coupling between trauma implants such as intramedullary nails and joint replacement constructs including total hip stems, total knee femoral components, total shoulder stems, reverse shoulder humeral components, and ankle arthroplasty tibial components. The modular configuration allows adaptability across anatomical regions, facilitating revision, limb salvage, and periprosthetic fracture stabilization.

### FIELD OF THE INVENTION

The present invention relates to orthopedic surgical systems, and more particularly to modular implant systems configured to connect fracture fixation implants with joint arthroplasty components across multiple anatomical regions, including but not limited to the hip, knee, shoulder, elbow, and ankle.

### BACKGROUND OF THE INVENTION

Conventional orthopedic implants are typically designed as closed systems. Intramedullary nails, total hip arthroplasty stems, and total knee components are engineered independently, with limited or no modular compatibility. This isolation restricts surgeons' ability to stabilize complex peri-prosthetic fractures or utilize existing implants for staged reconstructions. The absence of a standardized docking mechanism complicates revision and limb-salvage procedures. The present invention addresses these limitations by introducing a mechanical docking interface capable of securely joining implants from differing classifications or manufacturers.

### SUMMARY OF THE INVENTION

The disclosed modular docking system features a clothespin-style tapered male adaptor and a fluted female receiver configured to achieve a secure, fatigue-resistant connection between fracture fixation and arthroplasty implants. The interface provides reversible, cross-platform coupling, allowing intraoperative assembly of hip, knee, shoulder, elbow, or ankle components with standardized taper geometries. Optional locking mechanisms, such as set screws, external collars, or interference fits, further enhance mechanical stability.

### BRIEF DESCRIPTION OF THE DRAWINGS

In the drawings, like reference numerals denote corresponding components throughout the several views. Each figure illustrates specific embodiments of the modular docking system as described below.

FIG. 1: Exploded view of the modular docking system (1-4), showing the alignment of the male fitting adaptor (1) with the female receiver (2). The adaptor body bridges implants while the set screw (3) and flutes (4) ensure rotational and axial stability.

FIG. 2: Cross-sectional view an adaptor (5) with a female receiver (2) showing internal flutes (4) for anti-rotation and axial engagement depth for the male adaptor.

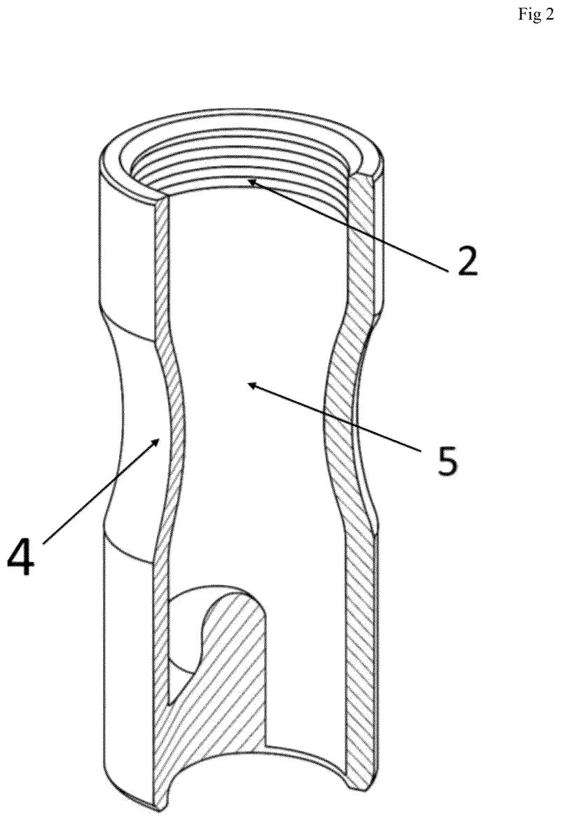

FIG. 3: Cross-sectional view of the adaptor (5) with a male ending (1) illustrating the clothespin-style longitudinal slot and taper angle of 1.5-3.0° per side, permitting radial compression on insertion.

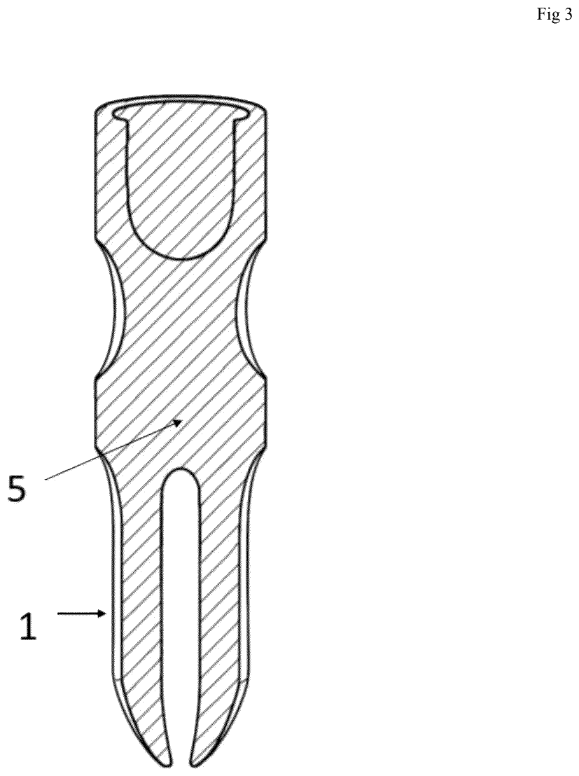

FIG. 4: Configuration showing orthogonal set screw (3) providing rigid fixation between adaptor (5) with male (1) and female (2) end, and a receiver.

FIG. 5: Variable adaptor configurations (5) of female (2) and male (1) components enabling cross-system coupling through standardized taper geometries.

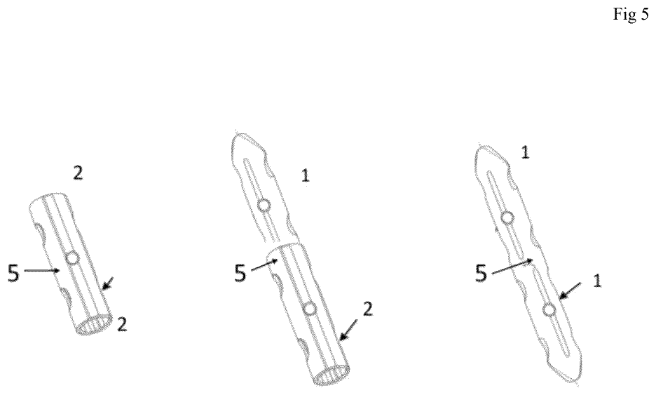

FIG. 6: Hip nail (10) with integrated male component (1) or female receiver (2) allowing cross-platform coupling while maintaining axial alignment.

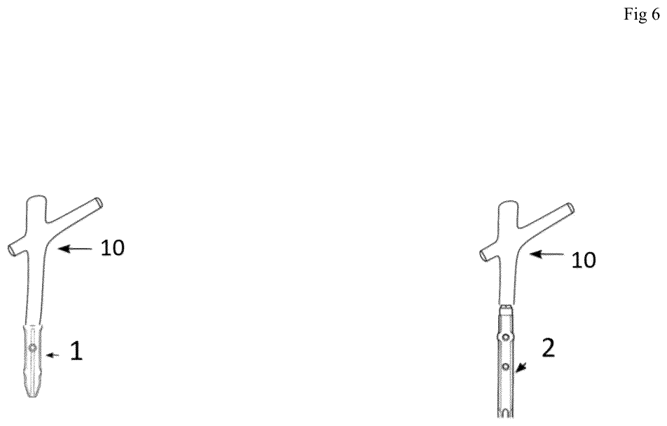

FIG. 7: Hip nail (10) docking directly into retrograde femoral nail (16) via male (1) and female (2) interfaces without an adaptor, maintaining torsional stability.

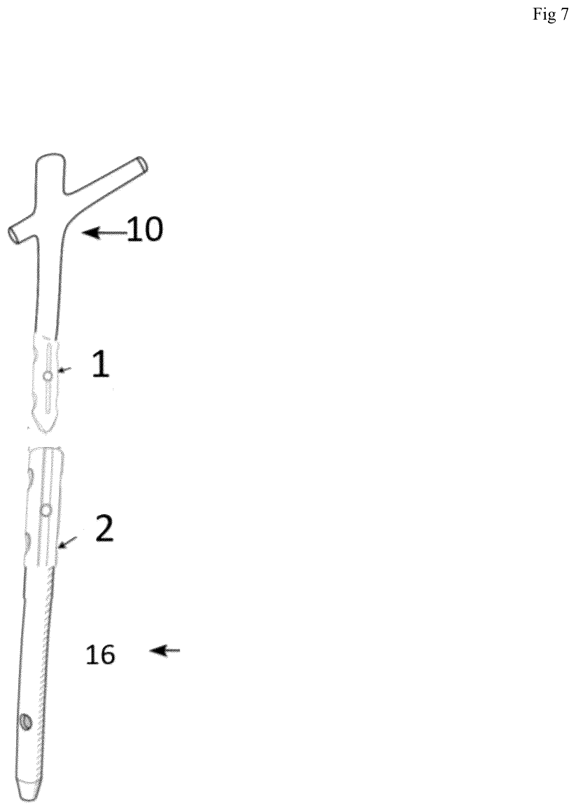

FIG. 8: Male-to-male interface of a hip nail (10) to a retrograde femoral nail (16) bridged by a female adaptor (2) with adaptor body (5), connecting dual male geometries (1).

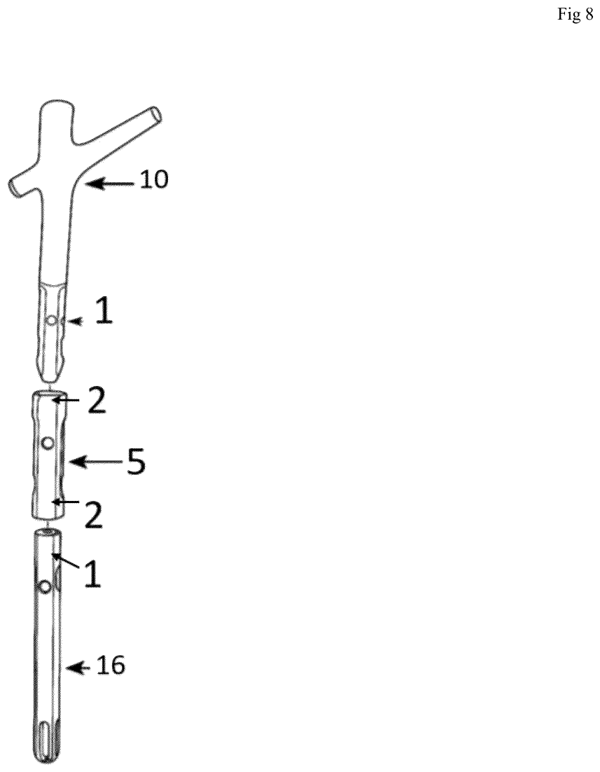

FIG. 9: Total hip arthroplasty stem (9) with male adaptor (1) engaging a retrograde femoral nail (16) with female receiver (2).

FIG. 10: Complete hip stem (9) to retrograde femoral nail (16) construct showing the separated hip stem and docked construct.

FIG. 11: Humeral nail (18) with dual docking configurations (1, 2) compatible with upper extremity arthroplasty stems.

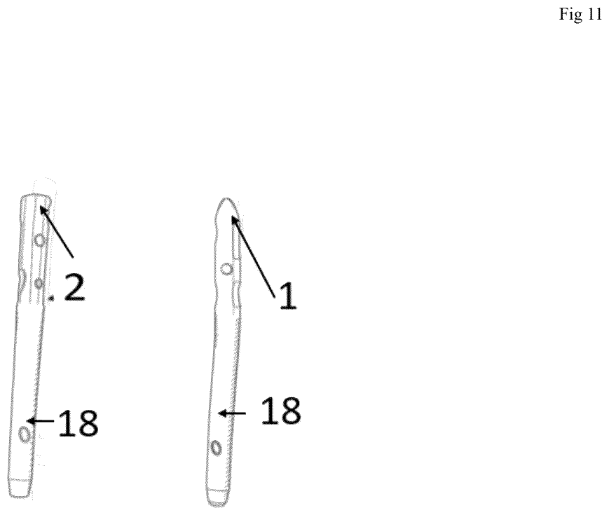

FIG. 12: Standard most common embodiment of a clothespin-style tapered fit showing split male adaptor (1) and fluted female receiver (2).

FIG. 13: Threaded male (1) and female (2) components providing torque-resistant mechanical coupling.

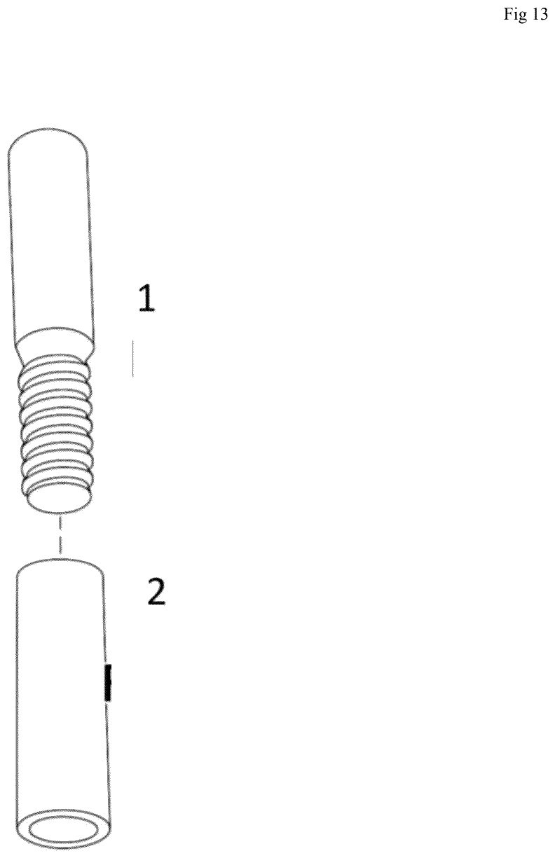

FIG. 14: Threaded male (1)-to-male (1) adaptor (5) coupling dual male ends with balanced stress distribution.

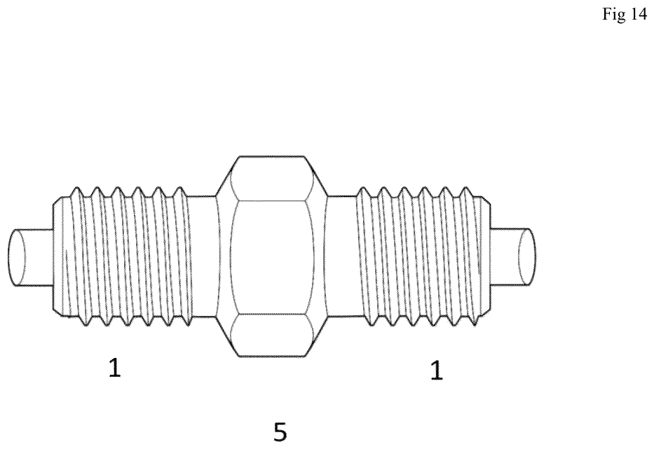

FIG. 15: Smooth interference fit between male (1) and female (2) components relying on surface friction and taper geometry.

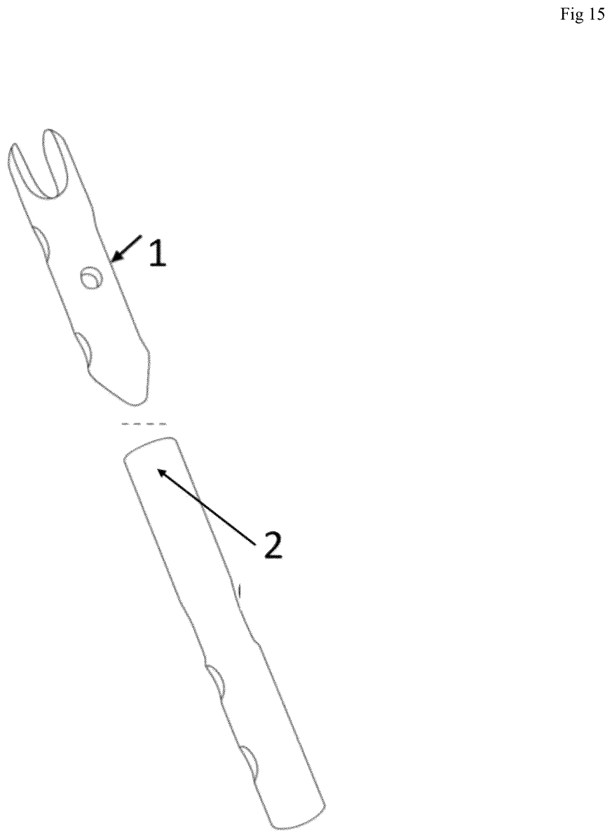

FIG. 16: Morse taper fit providing self-locking conical engagement for axial and torsional stability.

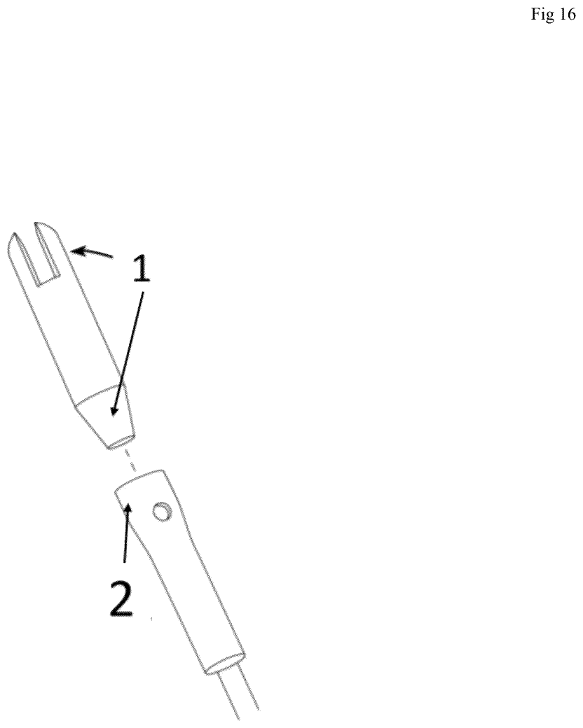

FIG. 17: Offset adaptors (13) enabling non-linear coupling up to 45° to accommodate anatomical alignment constraints.

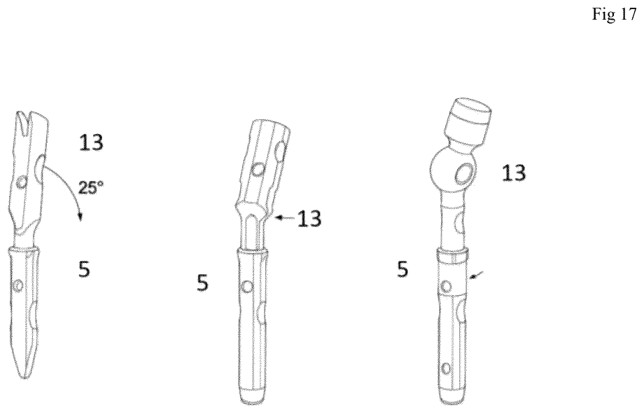

FIG. 18: Cannulated docking variant with guidewire channel (6) for alignment and cement delivery.

FIG. 19: External collar (17) for adjustable axial compression and locking at the docking interface of male (1) and female (2) receiver.

FIG. 20: Radiopaque markers (7) delineating docking boundaries under fluoroscopic imaging and an adaptor body (5).

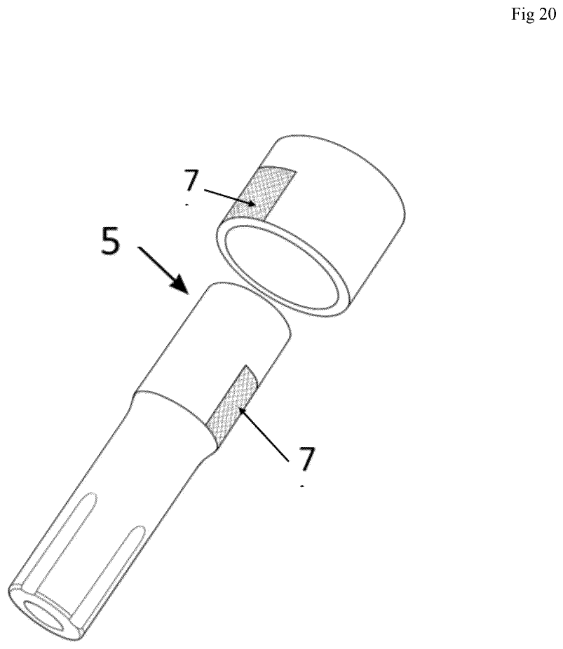

FIG. 21: Polymer insert (8) between metallic components of an adaptor (5) with male (1) and female (2) ends improving fatigue resistance and reducing corrosion.

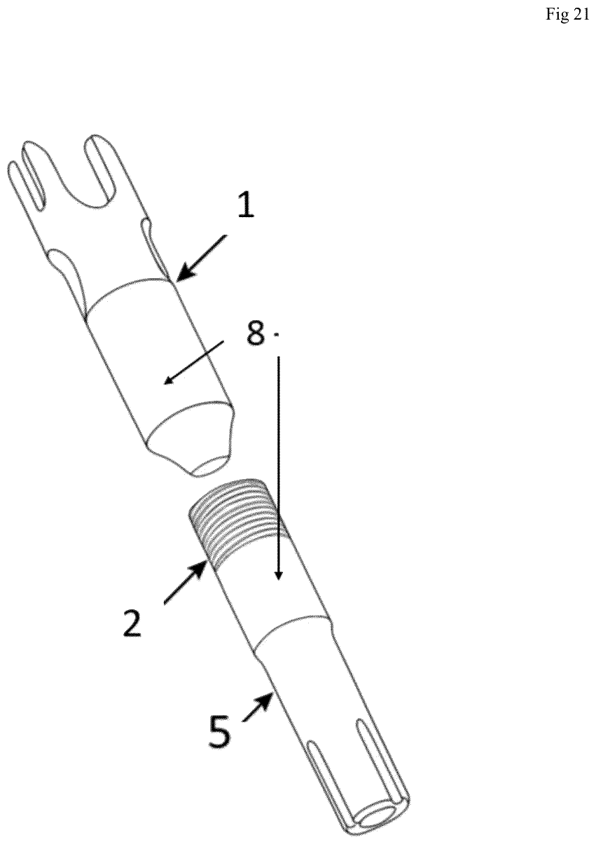

FIG. 22: Expandable locking element (12) in deployed configuration engaging internal bone surfaces.

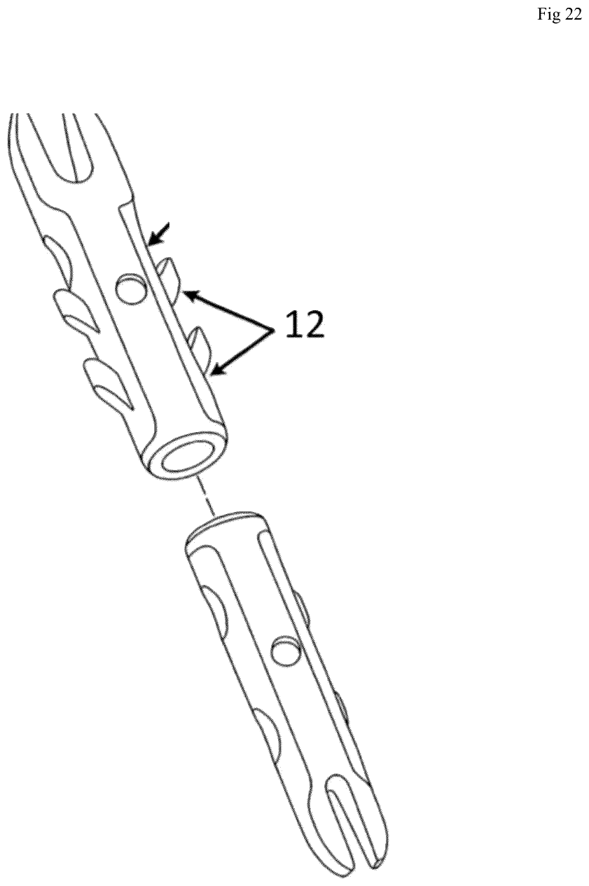

FIG. 23: Expandable locking element (12) in undeployed state prior to insertion.

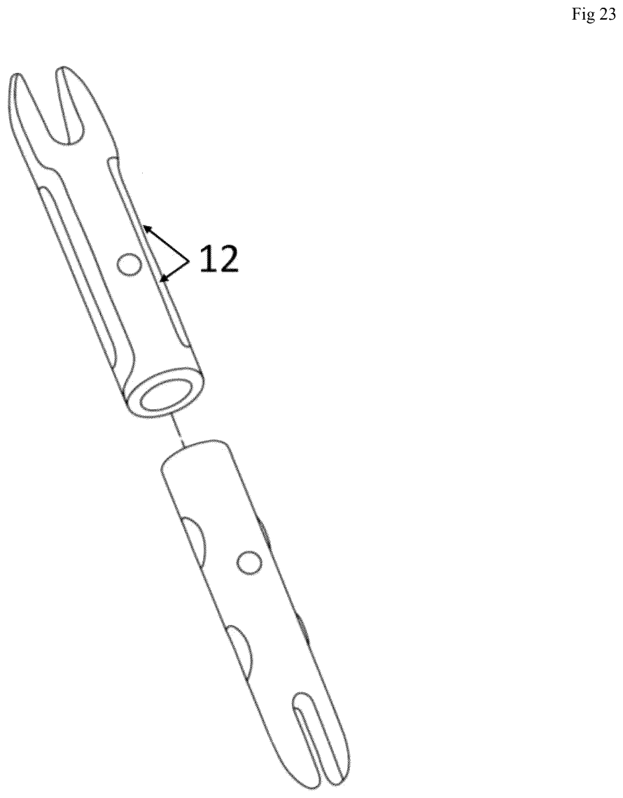

FIG. 24: Femoral nail (16) docked to total knee femoral component (9) via male (1) and female (2) interfaces.

FIG. 25: Reverse shoulder arthroplasty stem coupled to humeral nail (18) through male (1) and female (2) modular docking components on an attached adaptor (5).

FIG. 26: Total elbow arthroplasty stem docked to humeral nail (18) with male (1) and female (2) taper fit.

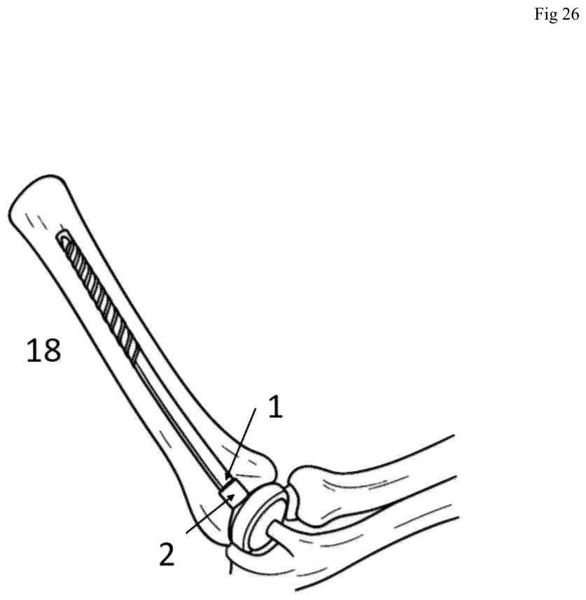

FIG. 27: Keyed anti-rotation adaptor (11) preventing torsional displacement using splined engagement.

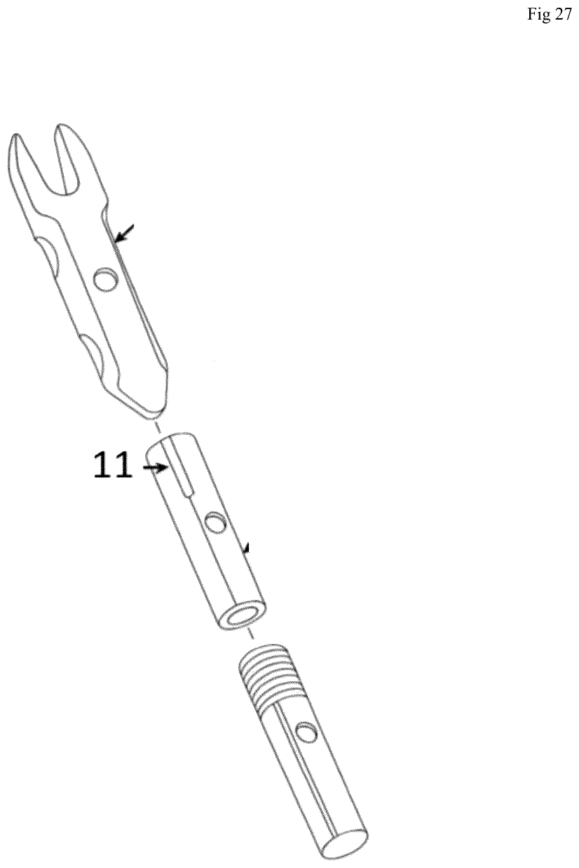

FIG. 28: Cannulated insertion instrument (14) aligning docking axis and applying impaction force.

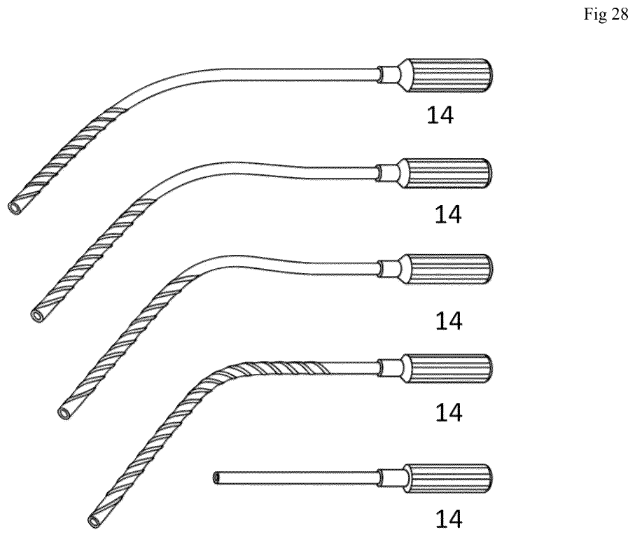

FIG. 29: Instrumentation (15) including alignment jig and impaction handle for modular assembly.

FIG. 30: Sterile packaged modular kit with adaptors (5), arthroplasty stems (9), and instruments (15) in tray format.

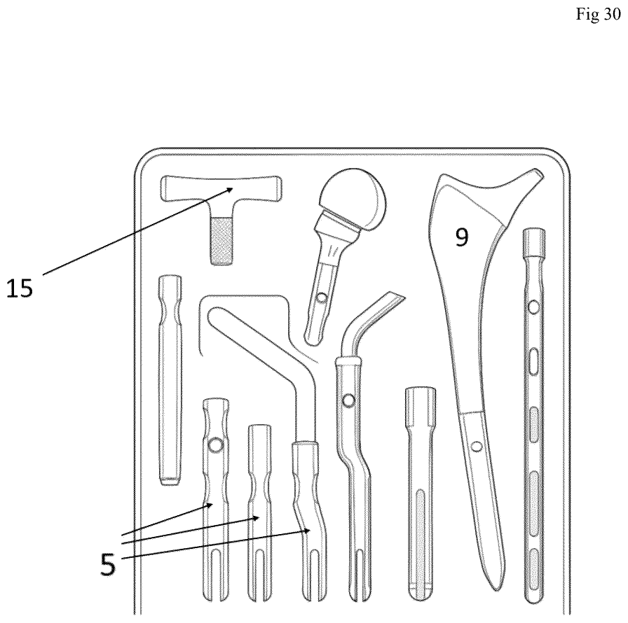

### DETAILED DESCRIPTION OF THE INVENTION

Referring to the drawings, the modular docking system enables cross-platform coupling between orthopedic implants such as intramedullary nails, total joint arthroplasty stems, and reconstructive components. The male adaptor (1) and female receiver (2) form the primary docking interface, characterized by axial taper compression and rotational stability provided by fluted splines. Optional set screws (3), collars (4), and keyed anti-rotation features (11) further enhance fixation integrity. Adaptors (5) may bridge implants with differing geometries, while offset connectors (13) accommodate anatomical variations. Cannulated designs (6) support guidewire-based alignment and injection of biologics or cement. The system allows modular intraoperative assembly and disassembly, reducing the need for full component revision during complex reconstructive procedures.

### FIGURE LEGEND APPENDIX

1—Male component (clothespin-style tapered stem)
2—Female component (fluted or splined docking socket)
3—Set screw (orthogonal or locking screw for anti-rotation)
4—Flutes
5—Adapter body (bridge between male/female interfaces)
6—Guidewire channel (central cannulation or passageway)
7—Radiopaque marker (contrast or visual indicator)
8—Polymer insert (hybrid interface or cushioning element)
9—Arthroplasty stem (hip, knee, shoulder, or elbow component)
10—Hip Fracture IM nail
11—Anti-rotation key (keyed spline or tab feature)
12—Expandable locking element (deployed anchor or expansion mechanism)
13—Offset adapter (angled or non-linear coupling element)
14—Instrument handle (detachable impaction or alignment handle)
15—Surgical jig (alignment or docking axis guide)
16—Retrograde Femoral Nail
17—External Collar
18—Humeral Nail

The modular docking system described herein is not limited to the femur or lower extremity. It is equally applicable to the upper extremity, including the shoulder and elbow, as well as to the ankle and foot. For example, a proximal humerus fracture with poor bone stock or failed fixation may be revised using a humeral stem with a male or female docking interface. This permits coupling to a reverse total shoulder baseplate or metaphyseal reconstruction component.

Similarly, in the elbow, distal humerus fractures or periprosthetic fractures around a total elbow arthroplasty can be managed using a humeral nail with a compatible docking interface, thereby allowing a stable, modular reconstruction without removal of well-fixed components. This approach reduces operative time and preserves host bone.

In the distal tibia or ankle, revision arthroplasty or trauma cases can employ tibial stems with docking capability to receive talar components, plates, or cement spacers. This platform modularity ensures extensibility of the system across anatomical regions.

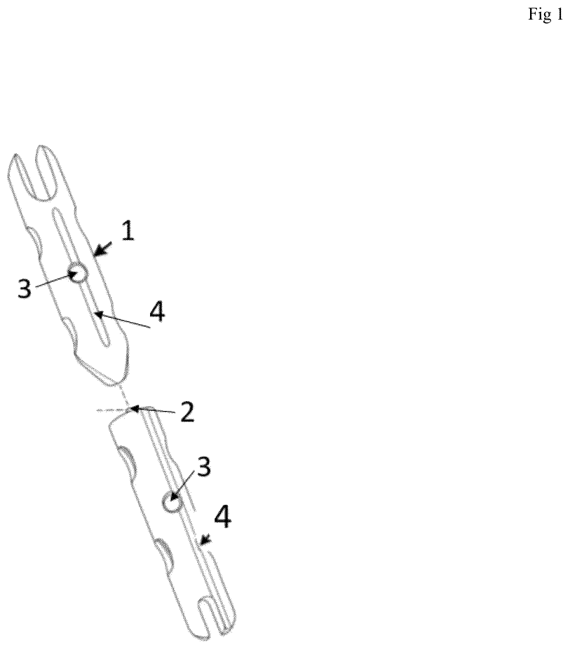

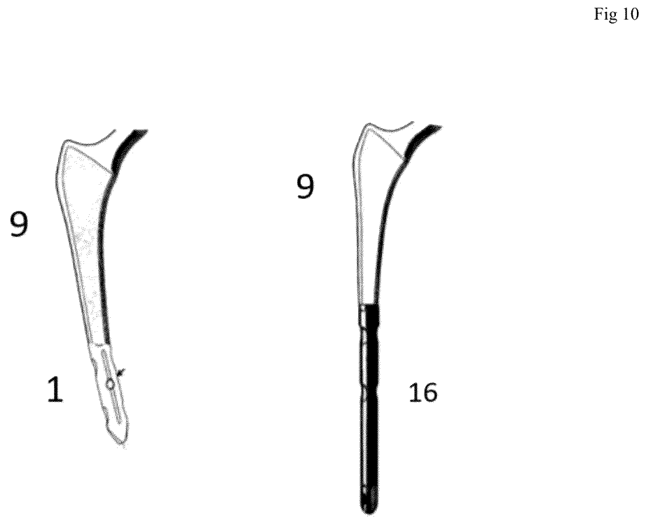

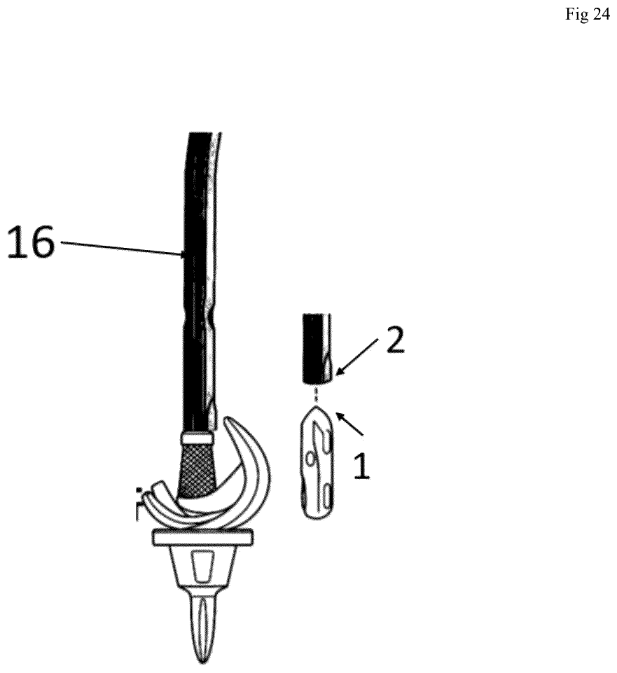

Referring now to the drawings, FIG. 10 shows a total hip prosthesis with a distal tapered stem designed to engage a fluted female receiver. FIG. 1 illustrates the male and female docking components, showing elliptical and fluted configurations designed for rotational and axial stability. FIG. 24 displays a total knee femoral component which can also house a female receiver, allowing for a modular connection with an antegrade femoral nail.

### Offset and Angled Adapter Embodiments

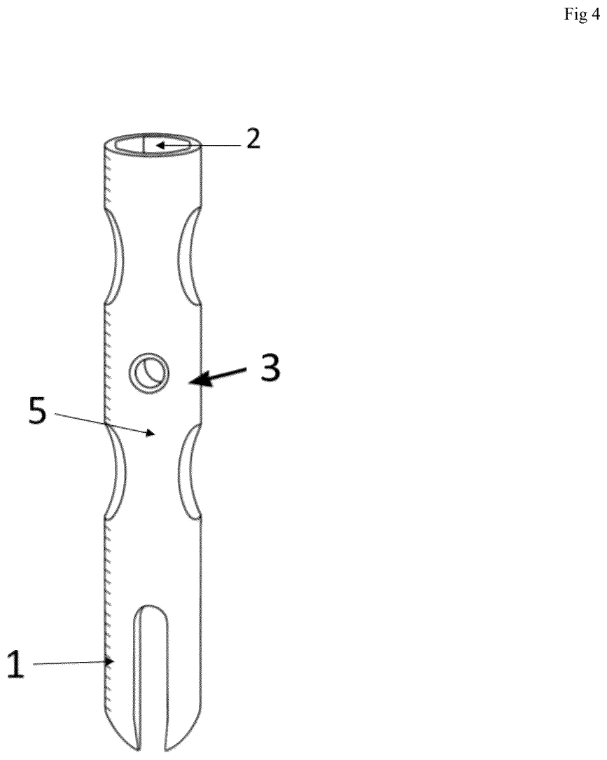

In some embodiments, an offset or angled adapter may be provided to accommodate anatomical deformities, pre-existing implants, or facilitate joint alignment. The adapter may include an oblique or curved pathway between the male and female docking interfaces, enabling non-linear coupling between implants. These adapters may include angular deviations ranging from 5° to 45°, or offset junctions with translational displacement up to 30 mm. Such embodiments allow for customized alignment, limb length restoration, or avoidance of compromised bone stock. The adapters may include splined, keyed, or set screw locking mechanisms to resist rotation and maintain axial stability under physiological loads. Materials and surface treatments used for these adapters shall be consistent with those described for the primary docking components. (see FIG. 4).

### Generalized Modular Coupling Embodiments

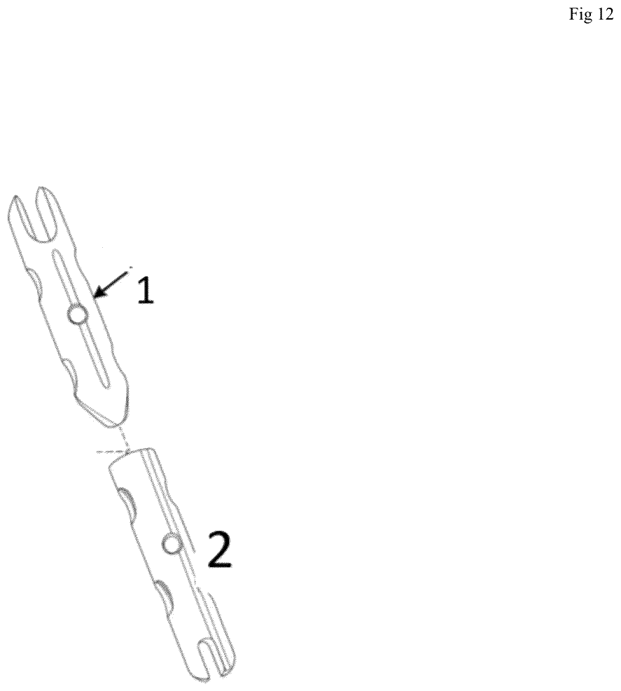

In further embodiments, the invention encompasses any modular interface that enables mechanical coupling between fracture fixation implants and arthroplasty components, regardless of specific interface geometry. The docking connection may include, but is not limited to, clothespin-style tapers, cylindrical interference fits, splined couplers, Morse tapers, dovetail geometries, or threaded junctions. The interface may be configured to provide axial alignment, rotational stability, fatigue resistance, and compatibility across implants from different systems or manufacturers. The invention broadly includes all such modular couplings intended to stabilize adjacent orthopedic components across anatomic zones. (see FIG. 12).

### Advanced Interface and Locking Variants

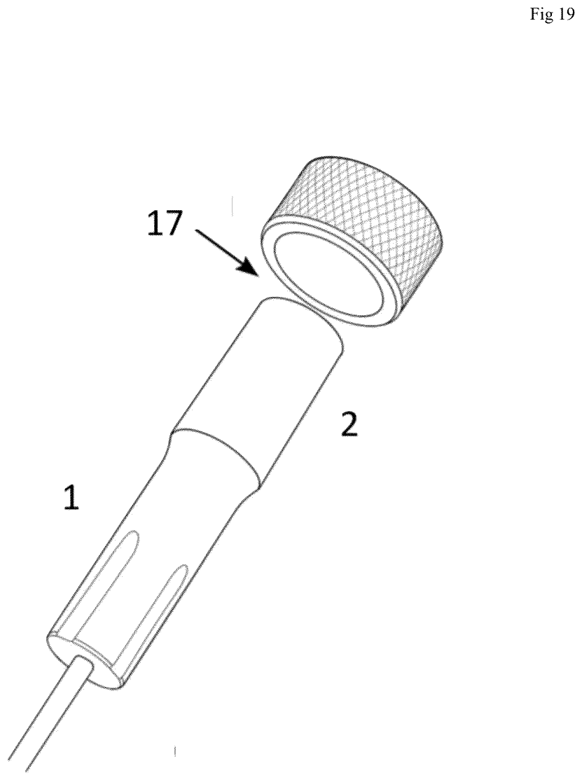

In further embodiments, the modular interface may include expandable components, keyed alignment features, or locking collars to enhance stability and rotational control. The interface geometry may also incorporate radiopaque markers or tactile engagement features to assist with intraoperative positioning. Materials for the male and female components may be differentially selected to achieve tailored stiffness, improved wear resistance, or optimized frictional characteristics. The coupling may further include a mechanical stop, indexed groove, or visual indicator to confirm full engagement during surgical assembly. (see FIG. 19).

### Reverse Threaded Interface Embodiments

In certain embodiments, the threaded components of the modular interface may include reverse (left-handed) threading on one or more ends. This may be used to prevent backout, control torque direction during insertion, or allow coupling of asymmetric implants with opposing thread orientations.

### Cannulated Interface Embodiments

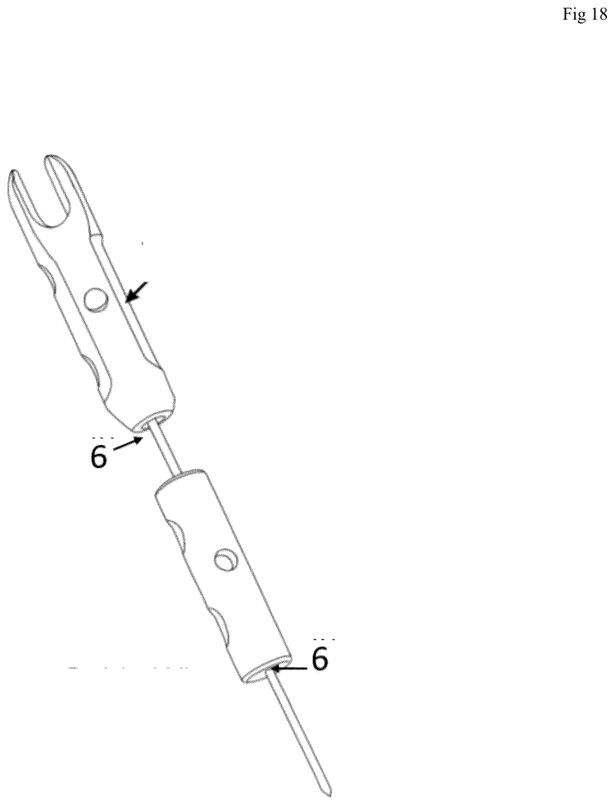

In various embodiments, the male and/or female components of the modular docking interface may be cannulated, allowing for passage of a guidewire to assist in intraoperative alignment and insertion. The cannulated bore may extend axially through the component and be configured to receive standard orthopedic guidewires (e.g., 3.0-4.0 mm), permitting accurate positioning during assembly of modular constructs. Cannulation may also facilitate cement injection, endoscopic navigation, or intraosseous delivery of biologics or augmentation materials. (see FIG. 18).

### Fracture-to-Fracture Implant Docking

In certain embodiments, the modular interface may be configured to connect two fracture fixation implants, such as an antegrade femoral nail and a retrograde femoral nail, thereby stabilizing a segmental or periprosthetic fracture through a continuous modular construct. The interface may comprise a male component integrated into one nail and a female receiver within the opposing nail, or a bridging adapter that mechanically couples like-interface configurations (e.g., male-male or female-female).

The clothespin-style male component comprises two flexible arms extending longitudinally and separated by a central axial gap. The outer surfaces are tapered to create radial compression upon insertion into the female receiver. Internal splines or flutes within the female receiver engage with grooves along the male arms to resist rotation. (see FIG. 12).

In further embodiments, the system includes dependent claims specifying: (a) the male taper length between 15-40 mm, (b) flute count of 4-8 splines at 90° intervals, and (c) locking set screws positioned orthogonal to the docking axis to secure rotational fixation. (see FIG. 4).

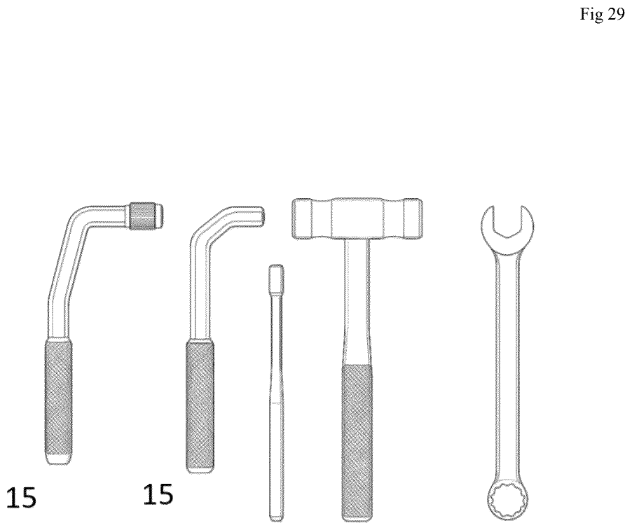

The docking interface may be inserted using specialized surgical instrumentation including an alignment jig, a calibrated impaction device, and an insertion handle. These tools facilitate co-axial alignment and ensure secure seating of the taper under controlled force, thereby reducing the risk of misalignment or toggle during implantation. (see FIG. 29)

All implants and components described herein, including the tapered male stems, fluted female receivers, and associated fixation hardware, are fabricated from biocompatible materials suitable for long-term implantation. Exemplary materials include titanium alloys (e.g., Ti-6Al-4V), cobalt-chromium alloys, stainless steel (e.g., 316L), and high-performance medical-grade polymers such as PEEK. Material selection may vary depending on the anatomical site, load-bearing requirements, and need for radiolucency.

In various embodiments, the modular interface permits interchangeability such that either the male or female component may be integrated into the arthroplasty stem or fracture fixation implant. This bidirectional configuration allows surgical flexibility depending on anatomical constraints or procedural preference, without departing from the scope of the invention.

### Surgical Use Case Examples

In one example, a patient presents with a peri-prosthetic fracture below a well-fixed total hip arthroplasty stem. Using the modular docking system, a retrograde femoral nail containing a female receiver is inserted distally, and the male component of the existing hip stem engages with the nail through the clothespin-style docking taper. This allows immediate stabilization without requiring revision of the arthroplasty component.

In another example, a patient requires limb salvage following tumor resection of the distal femur. A modular total knee femoral component containing a female docking interface is implanted, and a proximal femoral reconstruction nail with a male docking taper is aligned and secured, creating a mechanically stable reconstruction from hip to knee.

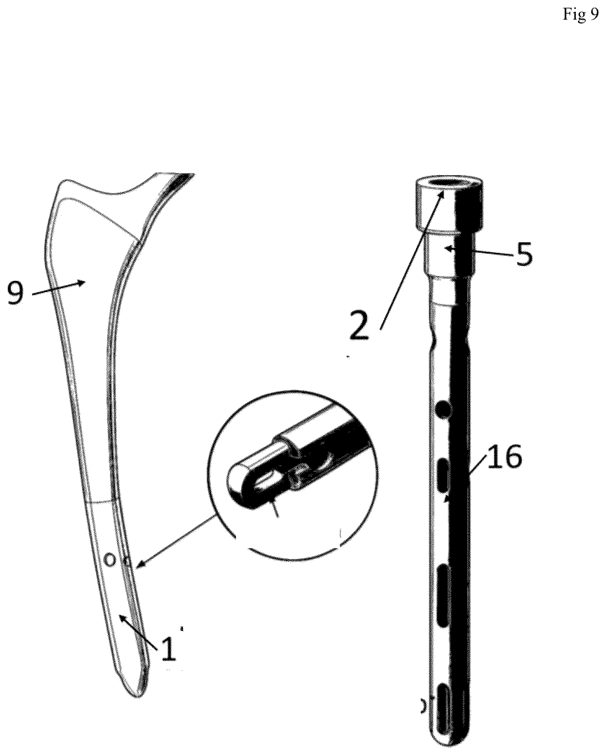

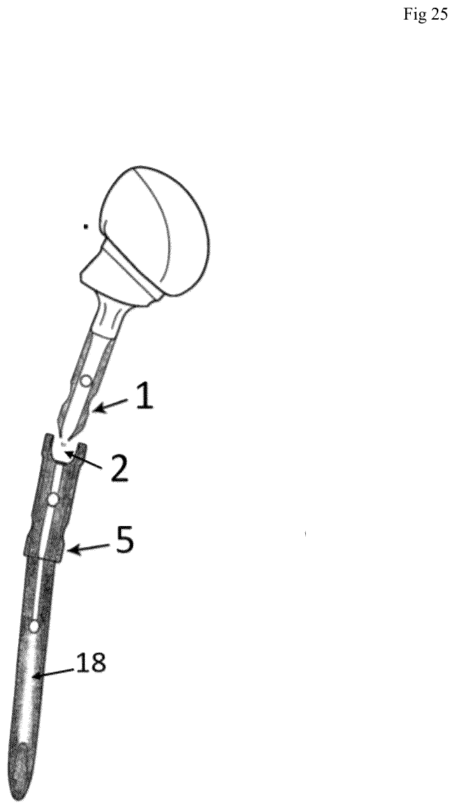

For upper extremity indications, a reverse shoulder arthroplasty stem may contain a female docking interface that receives a proximal humeral nail male component, enabling fracture stabilization and rotator cuff deficient arthroplasty in a single modular construct. (see FIG. 9 and FIG. 10). (see FIG. 25)

### Surgical Workflow Example—Hip to Knee Modular Salvage

Following stabilization of a subtrochanteric femur fracture with a proximally inserted intramedullary hip nail, intraoperative imaging reveals a previously undiagnosed periprosthetic fracture extending into the distal femur. Rather than explanting the nail or converting to a total femur replacement, the surgeon introduces a retrograde femoral nail with a female docking interface.

Through the same sterile field, the distal component is advanced into the femoral canal and engaged with the male taper of the proximal hip nail. A locking set screw is inserted through an access portal, securing the modular junction and restoring continuity across the fracture site. This approach avoids cement use, reduces operative time, and preserves the well-fixed proximal implant.

### Avoidance of Cemented Stem Revision in Periprosthetic Fractures

In cases where the arthroplasty component is cemented and remains well-fixed, surgical revision often requires extraction of the cement mantle-a process associated with increased operative time, risk of femoral perforation, blood loss, and potential loss of bone stock. The present invention eliminates the need for such revision by enabling distal fixation through a modular docking interface that engages the existing stem. This approach permits stabilization of periprosthetic fractures without disturbing the cemented prosthesis, thereby reducing surgical morbidity and preserving native bone.

In some embodiments, an adapter component may be provided to enable compatibility between like-interface implants, such as a male-to-male or female-to-female configuration. The adapter may comprise a dual-ended mechanical interface with a female receiver at one end and a male taper at the other, or a coupler with dual female or dual male geometries, thereby bridging otherwise incompatible docking surfaces. The adapter may include internal splines, locking features, or cement augmentation interfaces to maintain rotational and axial stability.

### BRIEF SUMMARY OF THE INVENTION

The present invention is directed to a modular orthopedic docking system that enables mechanical coupling between fracture fixation implants—such as intramedullary nails—and arthroplasty components, such as total joint replacement stems. A clothespin-style male stem and a fluted female receiver provide a secure, fatigue-resistant interface. The modular components may be integrated into or attached to total hip stems, total knee femoral components, total shoulder or reverse shoulder humeral stems, and ankle tibial components. The invention allows flexible intraoperative assembly, cross-platform compatibility, and improved treatment options for revision surgery, limb salvage, and periprosthetic fracture stabilization. (see FIG. 12).

### Comparative Analysis of Modular Docking Systems

Table 1 illustrates exemplary differences between the disclosed modular docking system and representative existing orthopedic implants, including standard intramedullary (IM) nails, conventional arthroplasty stems, and the Biomet Arcos Modular Femoral Revision System. These differences are non-limiting and serve to highlight the functional distinctions and novel capabilities of the present invention.

| | KlimaFix | | |
|---|---|---|---|
| | Modular | | Biomet |
| | Docking | Standard | Arcos |
| Feature | System | IM Nail | System |
| Clothespin-style | ✓ | X | X |
| male taper | | | |
| Fluted female | ✓ | X | ✓ |
| receiver for | | | |
| anti-rotation | | | |
| Cross-platform | ✓ | X | X |
| trauma-to- | | | |
| arthroplasty | | | |
| modularity | | | |
| Toggle resistance | ✓ | ✓ | ✓ |
| with axial stability | | | |
| Cannulated for | ✓ | ✓ | X |
| guidewire | | | |
| compatibility | | | |
| Reverse | ✓ | X | X |
| configuration | | | |
| compatibility | | | |
| Compatible with | ✓ | X | X |
| shoulder and | | | |
| ankle arthroplasty | | | |
| Revision-enabled | ✓ | X | ✓ |
| interface across | | | |
| implant classes | | | |

### PRIOR ART ANALYSIS AND DISTINCTIONS

Various modular orthopedic implant systems have been previously described, including modular femoral revision stems and joint arthroplasty constructs with taper-based or splined connections. Representative examples include the Biomet Arcos Modular Femoral Revision System (US20080071227A1), the S-ROM Modular Hip System (U.S. Pat. No. 4,911,693A), the Zimmer Biomet Compress Device (U.S. Pat. No. 6,533,799B1), the Stryker Restoration Modular System (US20100249815A1), and the Smith & Nephew Modular Femoral Components (US20060241582A1).

The following table summarizes key prior art references relevant to modular orthopedic implant systems. Each is compared against the present invention, highlighting distinguishing features that emphasize novelty and non-obviousness of the claimed docking system.

| | | | Key | Distinction from |
|---|---|---|---|---|
| Title | Patent No. | Assignee | Features | Present Invention |
| Biomet | US20080071227A1 | Biomet | Modular | No trauma- |
| Arcos | | Manufacturing | femoral | arthroplasty |
| Modular | | Corp. | stem with | cross-platform |
| Femoral | | | taper | docking; no |
| Revision | | | junctions | clothespin taper. |
| System | | | for | |
| | | | revisions | |
| S-ROM | U.S. Pat. No. 4,911,693A | DePuy | Tapered | No integration |
| Modular | | Orthopaedics, | modular | with retrograde |
| Hip System | | Inc. | junctions | nails or reverse |
| | | | in THA | configurations. |
| Zimmer | U.S. Pat. No. 6,533,799B1 | Zimmer, Inc. | Limb | Not modularly |
| Biomet | | | salvage | compatible with |
| Compress | | | prosthesis | IM nails or |
| Device | | | with | arthroplasty |
| | | | compressive | stems. |
| | | | fixation | |
| Stryker | US20100249815A1 | Stryker | Fluted | No docking with |
| Restoration | | Corporation | modular | trauma nails; lacks |
| Modular | | | revision | bidirectional |
| System | | | stems | interchangeability. |
| Smith & | US20060241582A1 | Smith & | Modular | No cross- |
| Nephew | | Nephew | tapers for | platform or |
| Modular | | | femoral | clothespin-style |
| Femoral | | | revision | docking |
| Components | | | | interface. |

### Biomechanical Validation Strategy

While no biomechanical testing has yet been performed, bench studies are planned to evaluate the resistance to implant toggle, fatigue failure, and axial loading across modular junctions. It is anticipated that the proposed docking geometries-particularly the clothespin-style taper, Morse taper, and spline interfaces-will provide improved torsional stability and resistance to mechanical migration. These theoretical advantages are based on established principles of press-fit interference, radial compression, and rotational keying. Future studies are intended to validate these anticipated outcomes using cadaveric models and fatigue cycling protocols. (see FIG. 12).

In some embodiments, biomechanical testing of the modular docking interface has demonstrated resistance to toggle and axial displacement under cyclical loading.

Preliminary benchtop simulations suggest toggle resistance exceeding 50 N·mm under a 1,000-cycle torsional stress profile using synthetic femora. Future in vitro cadaveric and in vivo animal studies are planned to validate fatigue resistance and axial stability under physiologic loads.

### Model-Based Performance Enhancement

Finite element analysis and theoretical modeling support that the modular docking system reduces mechanical toggle, particularly in short-stemmed constructs such as hip nails or humeral nails. The taper junction distributes axial and torsional loads more evenly along the interface, minimizing peak stress at the cortical bone-implant junction.

Comparative modeling further demonstrates that the modular junction attenuates stress risers and avoids sudden changes in stiffness, thereby decreasing the risk of periprosthetic fracture and fatigue failure over time. The ability to intraoperatively dock trauma and arthroplasty implants promotes construct continuity, even in the presence of compromised host bone.

### STATEMENT OF COMPLIANCE

This document is submitted as a Substitute Specification under 37 C.F.R. § 1.125. It replaces the previously filed specification in its entirety. The present version is properly ordered, double-spaced, and includes the expanded Brief Description of Drawings and Figure Legend Appendix. No new matter has been introduced.

1. A modular orthopedic docking system comprising a first implant having a tapered male component; and a second implant having a complementary tapered female receiver for mating with the male component to form a secure mechanical and axial connection; where the implants are composed of biocompatible materials suitable for long-term implantation, such as titanium alloys, cobalt-chromium alloys, stainless steel, or medical-grade polymers.

2. The system of claim 1, where the first implant is a total hip arthroplasty stem and the second implant is a retrograde femoral nail.

3. The system of claim 1, where the first implant is a hip fracture nail stem and the second implant is a total knee femoral component.

4. The system of claim 1, where the first implant is a reverse shoulder replacement stem and the second implant is a humeral nail.

5. The system of claim 1, where the first implant is a total shoulder replacement stem and the second implant is a humeral nail.

6. The system of claim 1, where the first implant is an ankle arthroplasty tibial stem and the second implant is a distal tibial nail.

7. The system of claim 1, where the female receiver comprises internal flutes or splines to resist rotational migration.

8. The system of claim 1, where the male component includes flexible arms forming a clothespin configuration to enable radial compression during insertion.

9. The system of claim 1, where the docking interface permits cement augmentation for additional fixation.

10. A method of surgically reconstructing a fractured or failed femur using the docking system of claim 1, comprising implanting the first implant into the proximal or distal segment of the femur; implanting the second implant into the opposing segment; and coupling the two implants using the docking interface; where the implants are composed of biocompatible materials suitable for long-term implantation.

11. The system of claim 1, where the male and female components are interchangeable between the arthroplasty stem and the fracture fixation implant, such that either component may serve as the proximal or distal docking element.

12. The system of claim 1, further comprising an adapter for mechanically coupling two implants having like interface geometries, where the adapter includes at least one male and one female docking interface, or a dual-male or dual-female configuration.

13. The system of claim 1, wherein the male and female components are configured to enable mechanical compatibility between implants produced by different manufacturers or modular systems through standardized taper dimensions or adapter interfaces.

14. The system of claim 1, further comprising an adapter having an angled or offset docking interface, where the male and female components are coupled via a non-linear pathway to accommodate anatomical or implant-specific alignment constraints.

15. A modular orthopedic interface comprising a mechanical coupling configured to connect a fracture fixation implant and an arthroplasty component, where the interface comprises a geometry selected from the group consisting of: a tapered interference fit, a threaded coupling, a fluted spline interface, a Morse taper, or a clothespin-style taper; and where the coupling enables intraoperative modular assembly between implants intended for reconstruction or stabilization across adjacent anatomic regions.

16. The system of claim 15, where the mechanical coupling comprises an angled or offset adapter for accommodating anatomical deformities or alignment needs.

17. The system of claim 15, where the modular coupling includes components sourced from different implant manufacturers.

18. The system of claim 15, where the interface comprises a non-threaded interference fit or a threaded coupling.

19. The system of claim 15, where the coupling interface permits intraoperative reversal of male and female components based on surgical preference.

20. The system of claim 15, where the modular coupling interface is for connecting two fracture fixation implants, including implants originating from different manufacturers or systems.

21. The system of claim 15, wherein one or more components of the modular coupling interface are cannulated to receive a guidewire for surgical alignment or delivery of augmentation materials.

22. The system of claim 15, where the threaded interface includes reverse threading on at least one male or female component to prevent loosening or enable asymmetric coupling.

23. The system of claim 15, wherein the modular coupling interface comprises at least one of the following: (a) an expandable locking element; (b) a keyed rotational alignment feature; (c) a radiopaque or visual alignment marker; (d) a differential material pairing; or (e) an external collar configured to enhance engagement stability.

24. A modular orthopedic docking system comprising a first implant including a tapered male component having at least two flexible longitudinal arms separated by an axial gap, forming a clothespin-style configuration; a second implant including a complementary fluted female receiver for receiving the male component with radial compression to form a secure axial and rotational mechanical connection; where the implants are composed of biocompatible materials suitable for long-term implantation.

25. The system of claim 1, wherein the docking interface is configured to permit intraoperative modular assembly and secure mechanical coupling between implants of differing classifications and/or manufacturers.

26. The system of claim 24, where the male component includes a taper angle between 1.5 and 3.0 degrees per side.

27. The system of claim 24, where the clothespin-style arms are configured to compress radially by at least 0.5 mm during insertion.

28. The system of claim 24, where the female receiver includes 4 to 8 internal splines spaced at approximately 90-degree intervals.

29. The system of claim 24, further comprising an orthogonal set screw for locking the male component against rotational displacement.

30. The system of claim 24, further comprising an orthopedic adapter having a first end with a tapered male docking component and a second end with a tapered female receiver.

31. The adapter of claim 30, where the first and second ends are connected via an offset or angled body portion providing a non-linear axis of coupling to accommodate anatomical alignment constraints or preexisting implants.

32. The system of claim 1, further comprising a surgical instrument including: a guide jig configured to align the docking axis between two implants; and a detachable handle configured to apply axial force to the male component for seating within the female receiver.

33. A method of stabilizing a periprosthetic femoral fracture below a total hip arthroplasty implant or hip fracture nail, comprising inserting a retrograde femoral nail having a female docking interface into the distal femur; engaging the male component of the existing hip stem with the nail via a clothespin-style tapered docking interface to form a mechanically locked construct.

34. A method of reconstructing a femur following tumor resection, comprising implanting a total knee femoral component having a female docking receiver; implanting a proximal femoral nail with a tapered male docking stem; coupling the two implants via a docking interface to span the femur with a modular construct

35. A method of reconstructing a proximal humerus in a patient requiring reverse shoulder arthroplasty, comprising inserting a reverse shoulder stem with an integrated female docking component; coupling the stem with a male-ended proximal humeral fracture nail; where the modular interface provides combined fixation and arthroplasty support.
